(19) United States
(12) Patent Application Publication
(10) Pub. No.: US 2023/0166118 A1
(43) Pub. Date: Jun. 1, 2023

(54) PORTABLE AED CASE

(71) Applicant: TRISULT HOLDING B.V., Venray (NL)

(72) Inventors: Ruben HALMANS, Venray (NL); Fredrik ANDERSSON, Kolbäck (SE)

(73) Assignee: TRISULT HOLDING B.V., Venray (NL)

(21) Appl. No.: 17/920,956

(22) PCT Filed: Apr. 26, 2021
(86) PCT No.: PCT/EP2021/060833

§ 371 Date: Oct. 24, 2022

(30) Foreign Application Priority Data
Apr. 27, 2020 (EP) 20171620.6

(51) Int. Cl. A61N 1/39

(52) U.S. Cl. CPC A61N 1/3968; A61N 1/3904; A61N 1/3925; A61N 1/3975

(57) ABSTRACT

A mobile case for an AED includes a device for regulating the temperature in order to keep the AED within its operational temperature range of 0 to 45° C. for at least 48 hours. The case may also include sensors controlling temperature, global position and the technical status of the AED as well as a communication device which allows for reporting the data of the AED and its position to a control center.

The invention pertains to a portable case for an automatic external defibrillator (AED).

Cardiac arrest is one of the most important causes of death and requires immediate conduction of proper first aid procedures. AEDs are indispensable tools for these procedures and their immediate use can significantly increase the chance of survival after a cardiac arrest. To date AEDs are available which cannot only be used by professionals but also by laypersons as they e.g. give acoustic instructions for use. For these reasons, AEDs are available at many public places such as railways stations, airports or important touristic sights. In order to make sure that an AED is working reliably if needed, proper storage of AEDs is indispensable. Typically, AEDs are stored in eye-catching cabinets in easily accessible places to make sure that in an emergency they are noticed, taken and used immediately.

To also assure proper functionality of an AED in emergency cases, proper storage of the AED is crucial. The operational temperature of an AED is 0-45° Celsius. For a reliable functionality care has to be taken that this temperature range is accurately kept. AEDs which have faced temperature outside that range cannot be expected to work reliably. Therefore, the temperatures inside the cabinet has to be controlled, regulated if necessary and temperature exceeding the above range, or failures of temperature regulation have to be reported to a control center in order to make sure that unreliable AEDs can be exchanged as soon as possible.

The same holds true for AEDs which suffer from an improper status of the device itself which can either result from a failed self-test or a low charge status of the battery. Therefore, the status of both the battery and the AED itself has to be checked automatically on a regular basis and reported at least in the case of a low battery status or failed self-test.

Use of an AED is not the only necessary first aid procedure in case of cardiac arrest. Both professional first aid and immediate hospitalization require rescue workers and an ambulance vehicle such as a cars, fire trucks, lorries, bikes, motorbikes, scooters, ambulances, trains, military vehicles, helicopters, airplanes, boats, namely SAR boats, to be at the place of emergency as soon as possible. In order to assure proper alert of rescue workers, AED cabinets are therefore equipped with a controlling device that reports that the cabinet has been opened causing immediate alarm in order to call rescue workers to the place of emergency.

All these requirements necessitate the AED cabinet to comprise a communication device which is able to report the status data of the AED and the temperature within the cabinet to a control center managing the AED and to also forward an alarm to rescue workers upon opening of the cabinet.

In order to fulfill these requirements, AED cabinets need constant electrical and communication connection and therefore have to be considered electronic devices as well.

As these functionalities require constant electrical connection, important features such as temperature control and regulation and immediate alert of rescue workers are not available for mobile applications.

A mobile application according to the present application is an application of an AED with is carried out without a wired connection to stationary electricity and/or communication networks for more than half an hour and in a context wherein the AED is moved by more than 250 meters. A typical characteristic of mobile application is that the AED is used further than 250 meters away from a wired power and/or communication network connection. This requires that the AED can be transported fast and easily with the required temperature conditions being fulfilled until the site of emergency is reached.

Fast and easy transport according to the pending application means that the AED case can be moved at a speed of at least 3.5 km/h by one single person over a distance of 250 meters or more using at most one hand and without use of any transporting means not being an entire part of the case.

As AEDs are indispensable tools for first aid in case of cardiac arrest, they are also an essential equipment of ambulance vehicles. Ambulance vehicles according to the application comprise both road and offroad vehicles but also aeroplanes like planes and helicopters as well as vessels such as SAR boats or lifeboats. For the time being, it is necessary to remove an AED from the ambulance vehicle if the vehicle is out of duty and it can be expected that the operational temperature range of the AED will be exceeded, e.g. due to environmental or weather conditions. In such occasions, it is necessary to store the AED in a place where it can be ensured that it is kept in its operational temperature range. Eventually, the AED has to be loaded back into the vehicle if duty of the vehicle is resumed. Being able to leave the AED in the ambulance vehicle also during its off-duty times would simplify the procedure of equipping an ambulance vehicle and eliminate sources of errors which, in case of emergency, can have fatal consequences.

It is the object of the invention to overcome this problem and to provide a human-portable AED case which overcomes the above-mentioned problems. The underlying object is being achieved by a portable case comprising a cavity containing an automatic external defibrillator (AED) characterized in that the cavity also comprises a means for removing thermal energy from the case within the case if the temperature overruns an upper threshold and for releasing thermal energy within the case if the temperature underruns a lower threshold. Throughout this application, the term “means for temperature regulation” will only be used for a device or material which actively adds thermal energy to the inside of the case either by transferring thermal energy from the outside of the case into the case or by converting another type of energy into thermal energy within the case or which actively removes thermal energy from the case either by transferring it to the outside of the case or by converting thermal energy within the case into another form of energy. Therefore, a means for temperature regulation will be a device or a material which is able to decrease the temperature within the case while the temperature outside the case is increasing and which is able to increase the temperature within the case while the temperature outside the case is decreasing.

Materials which decelerate exchange of thermal energy between the inside and the outside of the case due to their low thermal conductivity and/or their high heat capacity and/or their ability to reflect infrared radiation will throughout this application be called “thermally insulating materials”.

Thermally insulating materials of low heat conductivity can be expanded polymers such as expanded polystyrene (e.g. Styropor®), expanded polypropylene or expanded polyurethane, nonwovens or fabrics from synthetic or natural fibers such as glass wool or stone wool, or vacuum flasks. Thermally insulating materials of high heat capacity may be stones or water.

In an embodiment, a human-portable AED case comprises a thermally insulating material which facilitates regulation of the temperature within the case. The thermally insulating material may be a lightweight thermally insulating material, in order to make sure that it can be carried easily. Possible thermally insulating materials are expanded plastics such as expanded polystyrene known e.g. as Styropor® or expanded polypropylene. Also, other thermally insulating materials, such as expanded polyurethane or a vacuum flask, may be used. Thermally insulating materials with an ability to reflect infrared radiation may be surfaces which are either light colored or white colored or surfaces which also show high reflectivity for visible light such as metal surfaces, polished metal surfaces, surfaces made from e.g. aluminium, silver or other highly reflecting metals which might be coated with a transparent scratch protection layer such as a transparent polymer layer or a glass layer.

The case according to the invention comprises a cavity to take the AED which is surrounded by the thermally insulating material. The cavity may comprise means for fixation of the AED such as straps or belts. Furthermore, the cavity might comprise the means for temperature regulation, sensors, means for communication, means for indicating the position and/or means for electronic control of said devices. Said means may also be comprised in subcavities of said cavity. A subcavity according to the application is a cavity which is essentially connected to the cavity but formed by a recess in the wall of the cavity. Apart from the AED, the means for temperature regulation, temperature and position control and communication, also storage room may be available for further accessories which might be needed in incident of cardiac arrest, for example thermally sensitive drugs or devices which have to be at hand immediately in an emergency.

The thermally insulating material might either be rigid or flexible. The thermally insulating material may have different layers of different functionalities. In an embodiment, the thermally insulating material comprises one layer of material with low heart conductivity and one layer with high ability to reflect infrared radiation. In an embodiment, the thermally insulating material consists of two parts which may form a body and a lid and which may be connected by a hinge-joint, textile straps or any other connection means to allow for proper handling and to avoid loss of one of the parts of the case. In an embodiment, the lid and the body are also connected by means which allow to reversibly lock the lid in its closed position such that the case remains closed also under carrying or movement. Possible means for locking are hooks, clamps, mechanical locks, hook-and-loop fasteners such as Velcro® or zippers. In an embodiment, the case is dust and watertight.

The form of the thermally insulating material surrounding the cavity will determine the form of the case. In an embodiment, the outer shell of the thermally insulating material is covered with a covering shielding it from mechanical wear. The covering may be made from hard or soft shell material depending on the purpose of use of the case. The covering may furthermore be equipped with means for fixation around a human body as well as means for human carrying or other kind of movement by human. In an embodiment the means for fixation can be handles. In an embodiment the means for fixation can be plastic handles. In an embodiment the means for fixation can be telescope handles. In an embodiment the means for fixation can be hooks. In an embodiment the means for fixation can be plastic hooks. In an embodiment the means for fixation can be buckles. In an embodiment the means for fixation can be loops. In an embodiment the means for fixation can be textile loops. In an embodiment the means for fixation can be belts. In an embodiment the means for fixation can be textile belts. In an embodiment the means for fixation can be straps. In an embodiment the means for fixation can be textile straps. The means for fixation may allow for a variety of ways of fixation, carrying or moving by human. Depending on the covering material and the handling and fixation means, the case can e.g. take the form of a suitcase, a carrying bag for carrying by hand or over one shoulder comparable to a tote bag or portable on the back comparable to a rucksack. In an embodiment, the covering also comprises rolls giving the AED case the shape of a trolley.

In an embodiment, the human portable AED case comprises an access for external electric power supply. The access may allow for a complete removal of the power supply in order to make carrying as easy as possible. In order to reach this objective, the electric power access might be a socket. In an embodiment, the socket may be a low-voltage DC socket which allows for maximum compatibility with widely used DC supplying power systems such as car cigarette lighters, chargers for mobile devices or powerbanks. Further possible DC power sources may be mobile solar panels or fuel cells as used for charging e.g. mobile computers or telephones. The DC power supply may be 12 V. The DC power supply may be 6 V. Compatibility with these kinds of devices will allow for power supply to the AED case using as many opportunities as possible. Providing proper power adaptors, also the public AC network or mobile AC sources such as mobile wind turbines as available e.g. on sail boats may be used.

In an embodiment, the means for temperature regulation within the AED case is driven electrically. This may include an electrical heater for keeping the temperature inside the case above the lowest operating temperature of the AED. In an embodiment, the electrically driven means for temperature regulation is a thermoelectric transducer which can be used for both heating and cooling depending on the polarity of the voltage applied. Also use of a device based on cooling by expansion and warming by pressurization of a gas (Joule-Thomson effect) is possible.

In an embodiment, the means for temperature regulation is based on a phase-change material which absorbs thermal energy upon melting and releases thermal energy upon returning to the solid phase. The big advantage of phase-change materials is that they offer a means for temperature regulation which is not dependent on external energy supply, is reliable and completely reversible. In an embodiment the phase change material is encapsulated e.g. in plastic foil to form a handy package and is arranged in the cavity surrounded by the insulating material such that it can easily be removed and exchanged. This facilitates both exchange of damaged phase change material and use of different kinds of phase change materials with different phase change temperatures depending on the geographical region and the expected environmental condition such as weather and season of the year. Also, several handy packages of different phase change materials might be used at the same time depending on the range of external temperatures the case would have to be buffered against. In an embodiment, the chemical composition and the amount of phase change material and the material, the form and the thickness of the thermally insulating material are chosen such that the temperature of the AED can be kept within its operational temperature range for at least 48 hours without any electrical power supply. In an embodiment, the chemical composition and the amount of phase change material and the material, the form and the thickness of the thermally insulating material are chosen such that the temperature of the AED can be kept within its operational temperature range for at least 72 hours without any electrical power supply. In an embodiment, the case has thus no need for a permanent network-based power supply. In this constellation, an AED can easily be kept at its operational temperature over a weekend and therefore cover the usual off-duty time of an ambulance vehicle or enable availability of an AED for a mobile first aid team e.g. during a public event, such as a music festival or a sporting event, running over a weekend. Also, shorter outdoor excursions, for example leisure or research activities as well as military maneuvers in cold or warm regions could take advantage of the availability of an operational AED using the case according to the invention. In general, the case is intended for use by mobile first responders in a medical emergency which can be any kind of mobile person such as e.g. high way patrol officers, police forces, mountain rescue teams, doctors, firemen, lifeguards, military personnel, taxi drivers, search and rescue workers at sea, pilots or flight attendants.

In an embodiment, the phase change material is an organic phase change material based on paraffins, lipids, sugar alcohols or mixtures thereof. In an embodiment, the phase change material is an inorganic phase change material based on a salt hydrate such as sodium sulfate, sodium chloride, sodium nitrate, manganese nitrate, manganese chloride, potassium nitrate or mixtures thereof. In an embodiment, the phase change material is a hygroscopic material. In an embodiment, the phase change material is a solid-solid phase change material which absorbs and releases heat energy by means of a change in crystal structure. In an embodiment, the phase change material is based on ionic liquids.

In an embodiment, the case comprises a temperature sensor detecting the temperature in the cavity of the case and therefore the temperature, the AED comprised in the case is exposed to. In an embodiment, the temperature sensor is placed close to the AED and is connected to a device which alerts the user of the AED if the limits of the operational temperature range of the AED are approached or even exceeded. The sensor may be connected to the means for temperature regulation and control their operation. The combination of temperature sensor and alerting device can be an analog or digital thermometer showing the inner temperature of the case. In an embodiment, the temperature sensor is a digital thermometer and the alerting device gives an acoustic or optical signal of temperature range exceeding. In an embodiment, the signal of temperature range exceeding is not only given at the time of temperature range exceeding but indicates also if the operational temperature range of the AED had exceeded in the past and that therefore, the AED can no longer be considered to be reliably working. In an embodiment, the alerting device also indicates the degree and the time period of temperature range exceeding in order to give the operator a chance to estimate the degree of reliability still left for the AED.

In an embodiment, the cavity to take the AED also comprises means for indicating the position of the case. In an embodiment, the means for indicating the position of the case is a GPS antenna or a localization device using mobile network. In an embodiment the GPS antenna is connected to a display on the outer shell of the case and is hence able to indicate the geographical position to an operator allowing for giving rescue workers geographical coordinates in case of an emergency.

In an embodiment, the case comprises sensors for detection of optical or acoustic signals given by the AED about its own technical status such as self-test results and results of tests of the battery status. In an embodiment, these sensors are coupled to an electronic controller. Depending on whether the AED gives status signals optically, e.g. by light indicators, or acoustically, the sensors can e.g. be photoresists or microphones. In order to allow for compatibility with as many AED types as possible, several types of sensors may be present in the AED cavity. The electronic controller of the sensors may be programmed to the specific signals of different AED types. The status sensors might also be able to detect whether the AED is still present in the case or whether it has been removed. The AED does not need to be connected to the case by any kind of wired or wireless data connection in order to make the case compatible with as many types of AEDs as possible.

In an embodiment, the sensors or the electronic controller are connected to an indicator on the outer shell of the case which inform the operator about the status of the AED. This is of special importance as the materials used the case might not be transparent and many thermally insulating materials also have a potential of acoustic shielding. Hence, the status indications of the AED might not be noticed outside the case and therefore require to be forwarded to a proper indication device such as a display or a loudspeaker on the outside of the case giving at least an optical or acoustical alert for the case of a failure of the AED.

In an embodiment, the case comprises a means for communication such as a radio transmitter, a wireless network module, a low-power wide area network (LPWAN) module, or Bluetooth module or a module for communication via mobile network. Using this means, the values measured by the sensors comprised in the case such as the means for indicating the position, the temperature and the status of the AED can be transferred to a control center in order to control the usability of the AED from remote but also to get information about its removal from the case and its use in order to localize it for immediate information of rescue workers or for prosecution of possible theft of the AED and/or the case. In an embodiment, the communication device allows for a direct voice connection enabling e.g. a professional rescue worker to give immediate instruction for the case of an emergency.

In an embodiment, the case comprises a battery, which may be a rechargeable battery such as a lithium-ion battery, which is charged when the case is connected to electric power and which can drive the sensors for temperature control, the sensors for detection of the status of the AED, the means for detection of the position and the communication means in order to make sure that a proper communication with the control center and a reliable alert of rescue workers is possible. In an embodiment, the battery is able to keep up the functionalities of the sensors and communication devices longer than the combination of thermally insulating material and means for temperature regulation are able to keep the temperature inside the case in the operational temperature range of the AED in order to still be able to control the status of the AED and to communicate in a case of emergency.

1. A case containing an automatic external defibrillator (AED) said case comprising thermal insulation and a means for regulating the temperature within the case, wherein the case is portable.

2. The case according to claim 1 comprising an access for an external electric power supply.

3. The case according to claim 1 comprising sensors for detecting the temperature within the case.

4. The case according to claim 1 comprising sensors for detecting the technical status of the AED and/or the battery status of the AED and/or the removal of the AED.

5. The case according to claim 1 comprising a means for indicating the position of the case.

6. The case according to claim 1 comprising a means for communication.

7. The case according to claim 1 comprising a thermally insulating material surrounding a cavity within the case to take the AED and the means for thermoregulation.

8. The case according to claim 7 wherein the cavity comprises subcavities to take the sensors, the means for regulating the temperature and accessories needed in the incident of a cardiac arrest.

9. The case according to claim 1 wherein the means for regulating the temperature within the case is based on a phase-change material.

10. The case according to claim 1, wherein the means for regulating the temperature within the case is driven electrically.

11. The case according to claim 10 wherein the means for regulating the temperature within the case comprises a thermoelectric transducer.

12. The case according to claim 10 wherein the means for regulating the temperature within the case is based on cooling by expansion and warming by pressurization of a gas.

13. The case according to claim 1 comprising a battery.

14. The case according to claim 13 wherein the communication means is driven by the battery.

15. The case according to claim 13 wherein the means for indicating the position is driven by the battery.
